(19) United States
(12) Patent Application Publication
(10) Pub. No.: US 2025/0188514 A1
(43) Pub. Date: Jun. 12, 2025

(54) METHOD FOR DETERMINING THE ACTIVITY OF A DRUG OR DRUG CANDIDATE UNDER CONDITIONS OF MICROGRAVITY

(71) Applicants: KING FAISAL SPECIALIST HOSPITAL & RESEARCH CENTRE, RIYADH (SA); SAUDI SPACE AGENCY, RIYADH (SA)

(72) Inventors: Khalid S. ABU KHABAR, RIYADH (SA); Wijdan Abdulrahim ALAHMADI, RIYADH (SA)

(21) Appl. No.: 18/976,874

(22) Filed: Dec. 11, 2024

Related U.S. Application Data
(60) Provisional application No. 63/608,548, filed on Dec. 11, 2023.

(51) Int. Cl. C12Q 1/6809; C12N 5/0786; G01N 33/50; G01N 33/68

(52) U.S. Cl. CPC C12Q 1/6809; C12N 5/0645; G01N 33/5023; G01N 33/5047; G01N 33/6803; C12N 2503/02; C12N 2525/00; C12Q 2600/136; G01N 2500/10

(57) ABSTRACT

The present invention relates to a method for determining the activity of a drug or drug candidate and/or for predicting suitability of a drug or drug candidate for treatment or prevention of a disease and/or for predicting suitability of a drug or drug candidate for influencing ageing.

### CROSS REFERENCE TO A RELATED APPLICATION

This application claims the benefit of U.S. provisional application Ser. No. 63/608,548, filed Dec. 11, 2023, which is incorporated herein by reference in its entirety.

### FIELD OF THE INVENTION

The present invention relates to a method for determining the activity of a drug or drug candidate and/or for predicting suitability of a drug or drug candidate for treatment or prevention of a disease and/or for predicting suitability of a drug or drug candidate for influencing ageing.

### BACKGROUND OF THE INVENTION

Mimicking diseases and cellular responses associated with diseases in in-vitro model systems remains an important task that will not only allow the study of physiological and pathological process but also the identification of drugs that are useful in treating or preventing the respective disease. Likewise, ageing is a physiological process that is only poorly understood so far and for which in-vitro model systems would be highly desirable. There is therefore a need in the art to provide for a methodology that allows to determine the activity of a drug or drug candidate under conditions that mimick a disease or a set of diseases. There is also a need in the art to provide for a methodology that allows to predict the suitability of a drug or drug candidate for treating or preventing a disease or a set of diseases. There is furthermore a need in the art to provide for a methodology that allows to predict the suitability of a drug or drug candidate for exerting an influence on the ageing process.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a method for determining the activity of a drug or drug candidate and/or for predicting suitability of a drug or drug candidate for treatment or prevention of a disease and/or for predicting suitability of a drug or drug candidate for influencing ageing, said method comprising:
a) Providing, in any order but separate from each other, a first set of cells of either a monocytic cell line or of stimulated peripheral blood mononuclear cells (PBMCs), and at least one drug or drug candidate;
b) Optionally, stimulating an immune response of said first set of cells of said monocytic cell line or of said stimulated peripheral blood mononuclear cells (PBMCs);
c) Culturing said first set of cells of said monocytic cell line or of said stimulated PBMCs;
d) Subjecting said first set of cells to a condition of microgravity, and exposing said first set of cells of said monocytic cell line or of said stimulated PBMCs to said at least one drug or drug candidate, whilst continuing to subject said first set of cells to a condition of microgravity; or exposing said first set of cells of said monocytic cell line to said at least one drug or drug candidate, and subjecting said first set of cells to a condition of microgravity, whilst continuing to expose said first set of cells of said monocytic cell line to said at least one drug or drug candidate;
e) Stopping said first set of cells of said monocytic cell line being subjected to a condition of microgravity;
f) Determining RNA expression and/or protein expression of said first set of cells and compare said determined RNA expression and/or said protein expression of said first set of cells with RNA expression and/or protein expression, respectively, of a second set of cells of the same monocytic cell line or stimulated peripheral blood mononuclear cells (PBMCs) that have also been subjected to a condition of microgravity but that have not been exposed to said at least one drug or drug candidate;
wherein differences observed when comparing expression in said first set of cells and said second set of cells, in
   expression of RNAs or proteins involved in a biological pathway, and
   expression of RNAs or proteins associated with a particular disease, between said first set of cells and said second set of cells, are indicative of said drug or drug candidate being suitable for treatment or prevention of a disease associated with said biological pathway or said particular disease, respectively; and
wherein differences in
   expression of RNAs or proteins associated with ageing, between said first set of cells and said second set of cells, are indicative of said drug or drug candidate being suitable for influencing ageing.

In one embodiment, said monocytic cell line is a cell line selected from THP-1 monocytic cell line, U-937 monocytic cell line, and KG-1 monocytic cell line; and wherein said stimulated peripheral blood mononuclear cells (PBMCs) are peripheral blood mononuclear cells that have been stimulated by a stimulating agent selected from lipopolysaccharide (LPS), hemocyanines such as keyhole limpet hemocyanine, phorbol esters such as phorbol myristate acetate (PMA), granulocyte-macrophage colony-stimulating factor (GM-CSF), macrophage colony-stimulating factor (M-CSF), pro-inflammatory cytokines, such as TNFα, IFN-γ, IL-1ß, IL-6, and combinations of the foregoing.

In one preferred embodiment, said method comprises step b).

In one particularly preferred embodiment, step b) is performed by exposing said first set of cells to an antigen or stimulating agent, preferably selected from lipopolysaccharide (LPS), hemocyanines such as keyhole limpet hemocyanine, phorbol esters such as phorbol myristate acetate (PMA), granulocyte-macrophage colony-stimulating factor (GM-CSF), macrophage colony-stimulating factor (M-CSF), pro-inflammatory cytokines, such as TNFα, IFN-γ, IL-1ß, IL-6, and combinations of the foregoing.

In one embodiment, said condition of microgravity of step d) is a gravity in the range of from 1×10⁻⁶ g to 1×10⁻² g, with “1 g” being the standard gravity experienced on the surface of Earth.

In one embodiment, said subjecting said first set of cells to a condition of microgravity in step d) occurs over a period of from 6 hours to 10 days, preferably from 24 hours to 5 days, more preferably from 24 hours to 72 hours, even more preferably from 24 hours to 48 hours.

In one embodiment, said determining RNA expression of said first set of cells in step f) is performed by RNA-sequencing (RNA-Seq), and wherein determining protein expression of said first set of cells in step f) is performed by mass spectrometry, Olink® proteomics, Somalogic® proteomics, Luminex® proteomics and array-type proteomics.

In one embodiment, said disease is selected from inflammatory diseases, infectious diseases, cardiovascular diseases, neurological diseases, cancerous diseases, diseases of the kidney, diseases of the lung, and autoimmune diseases.

In one preferred embodiment,
said cardiovascular diseases are selected from coronary artery disease, heart failure, hypertensive heart disease, rheumatic heart disease, cardiomyopathy, dilated cardiomyopathy, myocardial infarction, arrhythmia, congenital heart disease, arteriosclerosis of coronary vessels, valvular heart disease, carditis, aortic aneurysm, peripheral artery disease, thromboembolic disease, venous thrombosis, pulmonary heart disease, endocarditis, and myocarditis;
said neurological diseases are selected from epilepsy, Alzheimer's disease, cerebral infarction, ischemic stroke, multiple sclerosis, Parkinson's disease, amyotrophic lateral sclerosis (ALS), ataxia, Bell's palsy;
said inflammatory diseases are selected from inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx, wherein preferably said inflammatory diseases are selected from the group comprising abscessation, acanthamoeba infection, acne vulgaris, actinomycosis, acute inflammatory dermatoses, acute laryngeal infections of adults, acute multifocal placoid pigment epitheliopathy, acute (thermal) injury, acute retinal necrosis, acute suppurative otitis media, algal disorders, allergic contact dermatitis, amyloidosis angioedema, ankylosing spondylitis, aspergillosis, atopic dermatitis, pseudorabies, autoantibodies in vasculitis, bacterial disorders, bacterial laryngitis, bacterial meningitis, Behçet's disease (BD), birdshot choroidopathy, Gilchrist's disease, Borna disease, brucellosis, bullous myringitis, bursitis, candidiasis, canine distemper encephalomyelitis, canine distemper encephalomyelitis in immature animals, canine hemorrhagic fever, canine herpes virus encephalomyelitis, cholesteatoma, chronic granulomatous diseases (CGD), chronic inflammatory dermatoses, chronic relapsing encephalomyelitis, chronic suppurative otitis media, Ocular Cicatricial pemphigoid (OCP), common upper respiratory infection, granuloma, Crohn's disease, cryptococcal disease, dermatomyositis, diphtheria, discoid lupus erythematosus (DLE), drug-induced vasculitis, drug or hypersensitivity reaction, encephalitozoonosis, eosinophilic meningoencephalitis, Erythema multiforme (EM), feline leukemia virus, feline immunodeficiency virus, feline infectious peritonitis, feline Polioencephalitis, feline spongiform encephalopathy, fibromyalgia, Fuchs Heterochromic Uveitis, gastroesophageal (laryngopharyngeal) reflux disease, giant cell arteritis, glanders, glaucomatocyclitic crisis, gonorrhea granular myringitis, Granulomatous meningoencephalitis (GME), herpes simplex, histoplasmosis, idiopathic diseases, idiopathic inflammatory disorders, immune and idiopathic disorders, infections of the immunocompromised host, infectious canine hepatitis, inhalation laryngitis, interstitial nephritis, irritant contact dermatitis, juvenile rheumatoid arthritis, Kawasaki's disease, La Crosse virus encephalitis, laryngeal abscess, laryngotracheobronchitis, leishmaniasis, lens-induced uveitis, leprosy, leptospirosis, leukemia, lichen planus, lupus, lymphoma, meningitis, meningoencephalitis in greyhounds, miscellaneous meningitis/meningoencephalitis, microscopic polyangiitis, multifocal choroiditis, multifocal distemper encephalomyelitis in mature animals, multiple sclerosis, Muscle Tension Dysphonia (MTD), mycotic (fungal) diseases, mycotic diseases of the CNS, necrotizing encephalitis, neosporosis, old dog encephalitis, onchocerciasis, parasitic encephalomyelitis, parasitic infections, Pars planitis, parvovirus encephalitis, pediatric laryngitis, pollution and inhalant allergy, polymyositis, post-vaccinal canine distemper encephalitis, prion protein induced diseases, protothecosis, protozoal encephalitis-encephalomyelitis, psoriasis, psoriatic arthritis, pug dog encephalitis, radiation injury, radiation laryngitis, radionecrosis, relapsing polychondritis, Reiter's syndrome, retinitis pigmentosa, retinoblastoma, rheumatoid arthritis, Rickettsial disorders, rocky mountain spotted fever, salmon poisoning disease (SPD), Sarcocystosis, sarcoidosis, schistosomiasis, scleroderma, Rhinoscleroma, serpiginous choroiditis, shaker dog disease, Sjogren's syndrome, spasmodic croup, spirochetal (syphilis) diseases, spongiotic dermatitis, sporotrichosis, steroid responsive meningitis-arteritis, Stevens-Johnson syndrome (SJS, EM major), epiglottitis, sympathetic ophthalmia, Syngamosis, syphilis, systemic vasculitis in sarcoidosis, Takayasu's arteritis, tendinitis (tendonitis), Thromboangiitis obliterans (Buerger Disease), tick-borne encephalitis in dogs, toxic epidermal necrolysis (TEN), toxocariasis, toxoplasmosis, trauma, traumatic laryngitis, trichinosis, trypanosomiasis, tuberculosis, tularemia, ulcerative colitis, urticaria (hives), vasculitis, vasculitis and malignancy, vasculitis and rheumatoid arthritis, vasculitis in the idiopathic inflammatory myopathies, vasculitis of the central nervous system, vasculitis secondary to bacterial, fungal, and parasitic infection, viral disorders, viral laryngitis, vitiligo, vocal abuse, vocal-cord hemorrhage, Vogt-Koyanagi-Harada syndrome (VKH), Wegener's granulomatosis, and Whipple's disease;
said infectious diseases are selected from viral infections, bacterial infections and protozoan infections, wherein, in particular, said infectious disease is selected from AIDS, Adenovirus Infection, Alveolar Hydatid Disease (AHD), Amoebiasis, Angiostrongyliasis, Anisakiasis, Anthrax, Babesiosis, Balantidiasis, Baylisascaris Infection, Bilharzia (Schistosomiasis), Blastocystis hominis Infection, Lyme Borreliosis, Botulism, Brainerd Diarrhea, Brucellosis, Bovine Spongiform Encephalopathy (BSE), Candidiasis, Capillariasis, Chronic Fatigue Syndrome (CFS), Chagas Disease, Chickenpox, Chlamydia pneumoniae Infection, Cholera, Chronic Fatigue Syndrome, Creutzfeldt-Jakob Disease (CJD), Clonorchiasis, Cutaneous Larva migrans (CLM), Coccidioidomycosis, Conjunctivitis, Coxsackievirus A16 (Cox A16), Cryptococcal disease, Cryptosporidiosis, West Nile fever, Cyclosporiasis, Neurocysticercosis, Cytomegalovirus Infection, Dengue Fever, Dipylidium caninum Infection, Ebola Hemorrhagic Fever (EHF), Alveolar Echinococcosis (AE), Encephalitis, Entamoeba coli Infection, Entamoeba dispar Infection, Entamoeba hartmanni Infection, Entamoeba polecki Infection, Pinworm Infection, Enterovirus Infection (Polio/Non-Polio), Epstein Barr Virus Infection, Escherichia coli Infection, Foodborne Infection, Aphthae epizooticae, Fungal Dermatitis, Fungal Infections, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis, Lassa Fever, Leishmaniasis, Visceral leishmaniasis (VL), Malaria, Marburg Hemorrhagic Fever, Measles, Meningitis, Mycobacterium avium Complex (MAC) Infection, Naegleria Infection, Nosocomial Infections, Nonpathogenic Intestinal Amebae Infection, Onchocerciasis, Opisthorchiasis, Papilloma virus Infection, Parvovirus Infection, Plague, Pneumocystis Pneumonia (PCP), Polyomavirus Infection, Q Fever, Rabies, Respiratory Syncytial Virus (RSV) Infection, Rheumatic Fever, Rift Valley Fever, Rotavirus Infection, Roundworms Infection, Salmonellosis, Scabies, Shigellosis, Shingles, Sleeping Sickness, Smallpox, Streptococcal Infection, Tapeworm Infection, Tetanus, Toxic Shock Syndrome, Tuberculosis, duodenum, Vibrio parahaemolyticus Infection, Vibrio septicemia, Viral Hemorrhagic Fever, Warts, Waterborne infectious Diseases, Varicella-Zoster Virus infection, Pertussis and Yellow Fever;
said cancerous diseases are selected from adenocarcinoma, choroidal melanoma, acute leukemia, acoustic neurinoma, ampullary carcinoma, anal carcinoma, astrocytoma, basal cell carcinoma, pancreatic cancer, Desmoid tumor, bladder cancer, bronchial carcinoma, estrogen dependent and independent breast cancer, Burkitt's lymphoma, corpus cancer, Carcinoma unknown primary tumor (CUP-syndrome), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic tumor, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymphomas, stomach cancer, malignant melanoma, malignant neoplasia, malignant tumors gastrointestinal tract, breast carcinoma, rectal cancer, medulloblastomas, melanoma, meningiomas, Hodgkin's/Non-Hodgkin's lymphoma, mycosis fungoides, nasal cancer, neurinoma, neuroblastoma, kidney cancer, renal cell carcinomas, oligodendroglioma, esophageal carcinoma, osteolytic carcinomas and osteoplastic carcinomas, osteosarcomas, ovarian carcinoma, pancreatic carcinoma, penile cancer, plasmacytoma, prostate cancer, pharyngeal cancer, rectal carcinoma, retinoblastoma, vaginal cancer, thyroid carcinoma, esophageal cancer, T-cell lymphoma, thymoma, tube carcinoma, eye tumors, urethral cancer, urologic tumors, urothelial carcinoma, vulva cancer, wart appearance, soft tissue tumors, soft tissue sarcoma, Nephroblastoma, cervical carcinoma, tongue cancer, invasive ductal carcinoma, invasive lobular carcinoma, ductal carcinoma in situ, lobular carcinoma in situ, small-cell lung carcinoma, non-small-cell lung carcinoma, bronchial adenoma, pleuropulmonary blastoma, mesothelioma, brain stem glioma, hypothalamic glioma, cerebellar astrocytoma, cerebral astrocytoma, neuroectodermal tumor, pineal tumors, sarcoma of the uterus, salivary gland cancers, anal gland adenocarcinomas, mast cell tumors, pelvis tumor, ureter tumor, hereditary papillary renal cancers, sporadic papillary renal cancers, intraocular melanoma, hepatocellular carcinoma, cholangiocarcinoma, mixed hepatocellular cholangiocarcinoma, squamous cell carcinoma, malignant melanoma, Merkel cell skin cancer, non-melanoma skin cancer, hypopharyngeal cancer, nasopharyngeal cancer, oropharyngeal cancer, oral cavity cancer, squamous cell cancer, oral melanoma, AIDS-related lymphoma, cutaneous T-cell lymphoma, lymphoma of the central nervous system, malignant fibrous histiocytoma, lymph sarcoma, rhabdomyosarcoma, malignant histiocytosis, fibroblastic sarcoma, hemangiosarcoma, hemangiopericytoma, leiomyosarcoma (LMS);
said diseases of the kidney are selected from chronic kidney disease, kidney stones, glomerulonephritis, polycystic kidney disease, nephrotic syndrome, end stage renal failure, renal cancer, interstitial nephritis, Fabry disease, focal segmental glomerulosclerosis (FSGS), lupus nephritis, IgA Nephropathy, cystinosis, complement 3 glomerulopathy, atypical hemolytic uremic syndrome, granulomatosis with polyangiitis, cardiovascular-kidney-metabolic syndrome. and minimal change disease;
said diseases of the lung are selected from asthma, chronic obstructive pulmonary disease (COPD), bronchiectasis, bronchitis, pulmonary fibrosis, sarcoidosis, lung cancer, pneumonia, COVID19, pulmonary edema, influenza, and pulmonary hypertension; and
said autoimmune diseases are selected from asthma, diabetes, rheumatic diseases, rejection of transplanted organs and tissues, rhinitis, chronic obstructive pulmonary diseases, osteoporosis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper IgE syndrome (HIES), Wiskott-Aldrich syndrome (WAS), ataxia-telangiectasia, immune mediated cancers, white cell defects, autoimmune diseases, systemic lupus erythematosus (SLE), rheumatoid arthritis (RA), multiple sclerosis (MS), immune-mediated or Type 1 Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, Goodpasture syndrome (GPS), myasthenia gravis (MG), Sympathetic ophthalmia, Phakogene Uveitis, chronical aggressive hepatitis, primary biliary cirrhosis, autoimmune hemolytic anemia, and Werlhof's disease.

In one embodiment, said influencing is selected from: preventing the onset of ageing, delaying the onset of aging, preventing the progress of ageing, delaying the progress of ageing, and combinations of the foregoing.

In a further aspect, the present invention also relates to a cell culture of monocytic cells or stimulated peripheral mononuclear blood cells (PBMCs) that have been cultured under a condition of microgravity. The monocytic cells, PBMCs and microgravity are preferably as defined herein.

In yet a further aspect, the present invention also relates to the use of such cell culture as a model for mimicking a disease or biological/biochemical pathway or a process of ageing.

In yet a further aspect, which may also be combined with the previous aspect, the present invention also relates to the use of such cell culture for determining the activity of a drug or drug candidate and/or for predicting suitability of a drug or drug candidate for treatment or prevention of a disease and/or for predicting suitability of a drug or drug candidate for influencing ageing.

### DETAILED DESCRIPTION

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

The term “drug”, as used herein, is meant to refer to a compound that is known to have a pharmacological effect when being administered to a patient.

The term “drug candidate”, as used herein, is meant to refer to a compound that is either suspected to have a pharmacological effect when being administered to a patient or that needs to be tested in order to establish whether it has a pharmacological effect.

The term “patient”, as used herein, is meant to refer to a human or an animal, preferably a human.

The term “microgravity” as used herein, is meant to refer to conditions of a gravity that is reduced in comparison to the gravity experienced by objects when being situated on the surface of the Earth. Typically “microgravity” is expressed as a fraction of “g”, “g” being the gravitational acceleration on the surface of the Earth, “g” being on average 9.81 m/s². In a preferred embodiment, a condition of microgravity is a gravity in the range of from 1×10⁻⁶ g to 1×10⁻² g.

The present inventor has surprisingly found that cultures of monocytic cell lines or cultures of stimulated peripheral blood mononuclear cells (PBMCs), when subjected to conditions of microgravity, represent an ideal disease model system which can be used to determining the activity of a drug or drug candidate and/or for predicting suitability of a drug or drug candidate for treatment or prevention of a disease. In particular preferred embodiments, the suitability and sensitivity of such model systems and methodology can be enhanced by stimulating an immune response in such monocytic cells or PBMCs, prior to subjecting them to microgravity and prior to exposing them a drug or drug candidate. Embodiments of the method of the present invention are particularly useful to identify drugs suitable for the treatment or prevention of a specific disease. Moreover, the present inventor has also surprisingly found that cultures of monocytic cell lines or cultures of stimulated peripheral blood mononuclear cells (PBMCs), when subjected to conditions of microgravity, represent an ideal model system for the process of ageing, and therefore such model system can be used to determining the activity of a drug or drug candidate and/or for predicting suitability of a drug or drug candidate for influencing ageing. Hence, the present invention opens up a whole new field for drug screening and drug repurposing.

### BRIEF DESCRIPTION OF THE FIGURES

The present invention is now further described by reference to the following figures.

FIG. 1 shows a flowchart of an embodiment of a method according to the present invention illustrating a protocol by which suitability of a drug for treatment or prevention of a disease can be determined. The drug may be taken from a drug library, and the whole library may be subjected to the protocol of the method.

FIG. 2 shows a flowchart of another embodiment of a method according to the present invention illustrating a protocol by which suitability of a drug for treatment or prevention of a disease can be determined.

FIG. 3 shows a flowchart of yet another embodiment of a method according to the present invention illustrating a protocol by which suitability of a drug for treatment or prevention of a disease can be determined.

FIGS. 4 and 5 show flowcharts of yet other embodiments of a method according to the present invention illustrating a protocol by which suitability of a drug for treatment or prevention of cardiological diseases and neurological diseases, respectively can be determined.

FIG. 6 shows a flowchart of yet another embodiment of a method according to the present invention illustrating a protocol by which suitability of a drug for treatment or prevention of dilated cardiomyopathy can be determined. The protocol also involves a virtual screening step the results of which may then be tested in real in an embodiment of the present invention.

FIG. 7 shows the results of performing a method according to embodiments of the present invention (i.e. employing conditions of microgravity) demonstrating various biological pathways and diseases which can be modelled (because they are enriched in appearance and can be observed) as inflammation downregulated genes during space microgravity in accordance with embodiments of the present invention.

FIG. 8 shows the results of performing a method according to embodiments of the present invention (i.e. employing conditions of microgravity) demonstrating various diseases which can be modelled (because they are enriched in appearance and can be observed) as inflammation downregulated genes during space microgravity in accordance with embodiments of the present invention.

FIG. 9 shows the results of performing a method according to embodiments of the present invention (i.e. employing conditions of microgravity) demonstrating various biological pathways and diseases which can be modelled (because they are enriched in appearance and can be observed) as inflammation upregulated genes during space microgravity in accordance with embodiments of the present invention.

FIG. 10 shows the results of performing a method according to embodiments of the present invention (i.e. employing conditions of microgravity) demonstrating various diseases which can be modelled (because they are enriched in appearance and can be observed) as inflammation upregulated genes during space microgravity in accordance with embodiments of the present invention.

FIG. 11 shows the results of performing a method according to embodiments of the present invention (i.e. employing conditions of microgravity) demonstrating various further biological pathways and diseases which can be modelled (because they are enriched in appearance and can be observed) as inflammation downregulated genes during space microgravity in accordance with embodiments of the present invention.

FIG. 12 shows the results of performing a method according to embodiments of the present invention (i.e. employing conditions of microgravity) demonstrating various further biological pathways and diseases which can be modelled (because they are enriched in appearance and can be observed) as inflammation upregulated genes during space microgravity in accordance with embodiments of the present invention.

FIG. 13 shows the results of performing a method according to embodiments of the present invention (i.e. employing conditions of microgravity) demonstrating various further diseases which can be modelled (because they are enriched in appearance and can be observed) as inflammation upregulated genes during space microgravity in accordance with embodiments of the present invention.

FIG. 14 shows various pathways that have been identified as being affected by performing embodiments of the present invention. These pathways have been grouped by cell process.

FIG. 15 shows a schematic overview of an example of an embodiment of a cellular model of inflammatory diseases and the way it is used. On the right is a typical flow chart, and on the left of the figure are responses that can be tested by such model, and conclusions can be drawn therefrom. It should be noted that this figure does not explicitly refer to the use of microgravity, but the methodology shown therein can be performed in the presence or absence of conditions of microgravity.

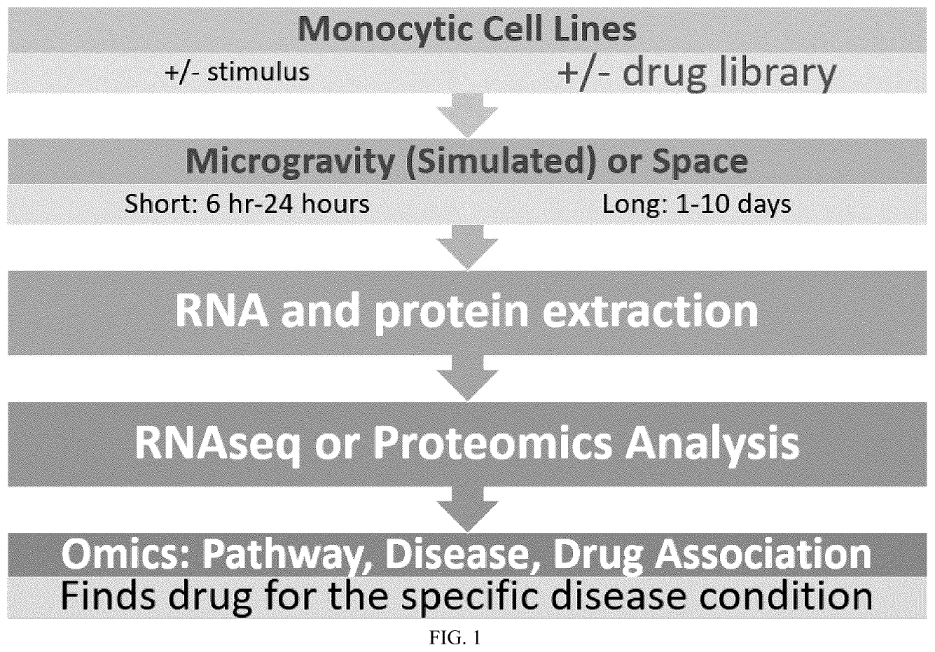

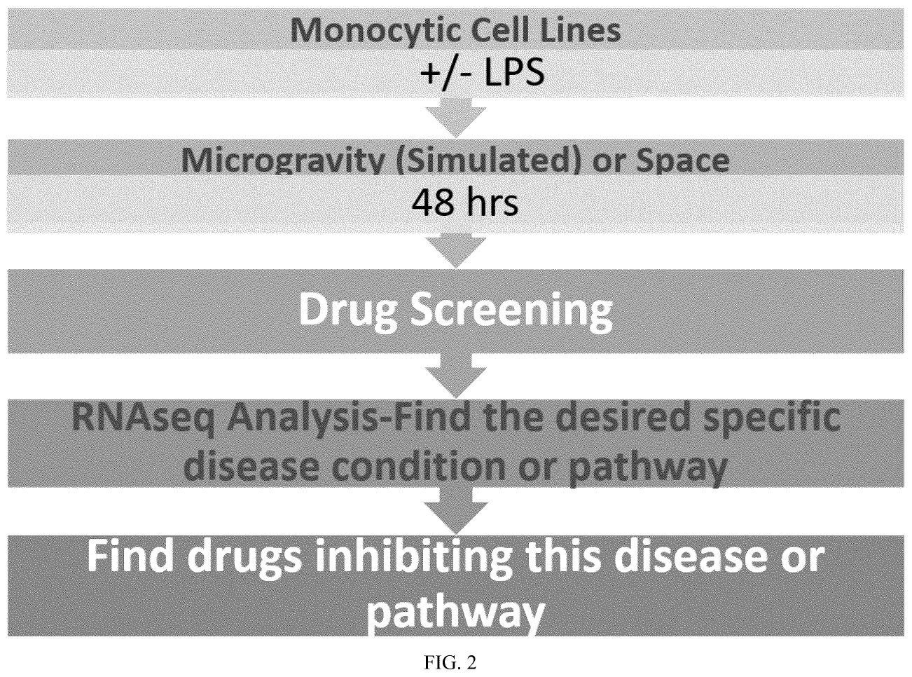

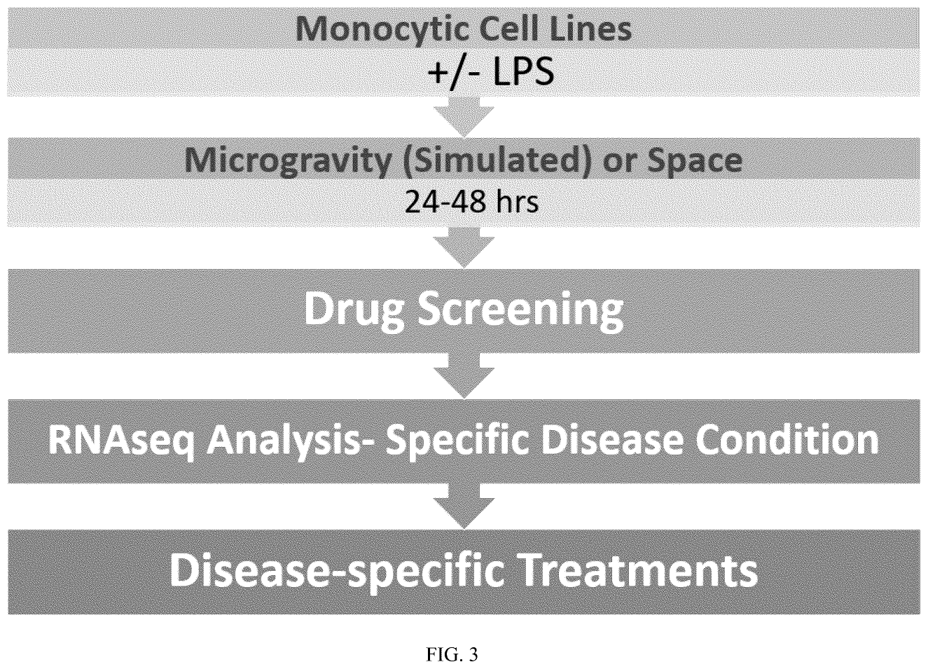

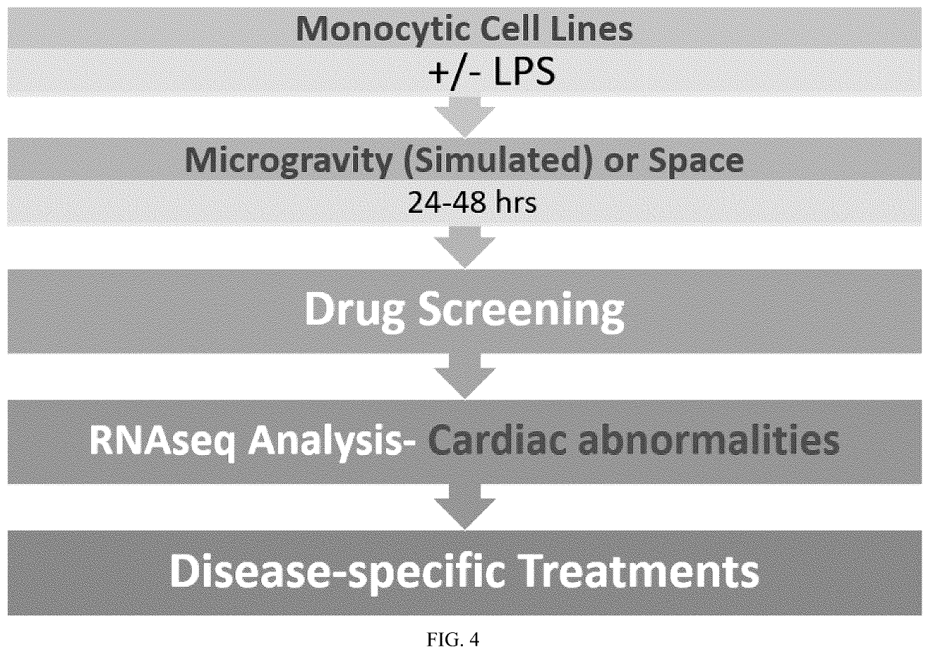

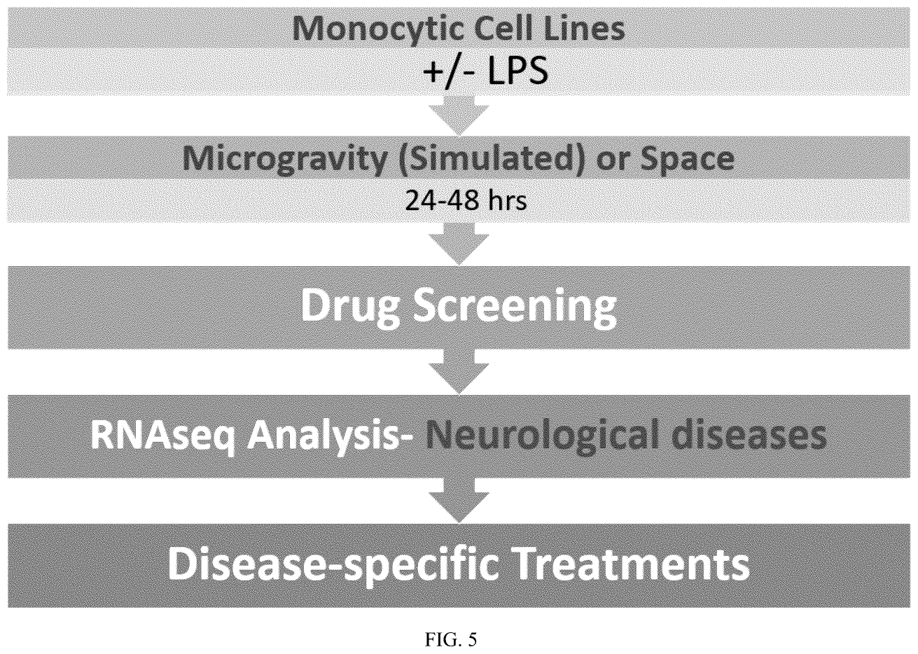

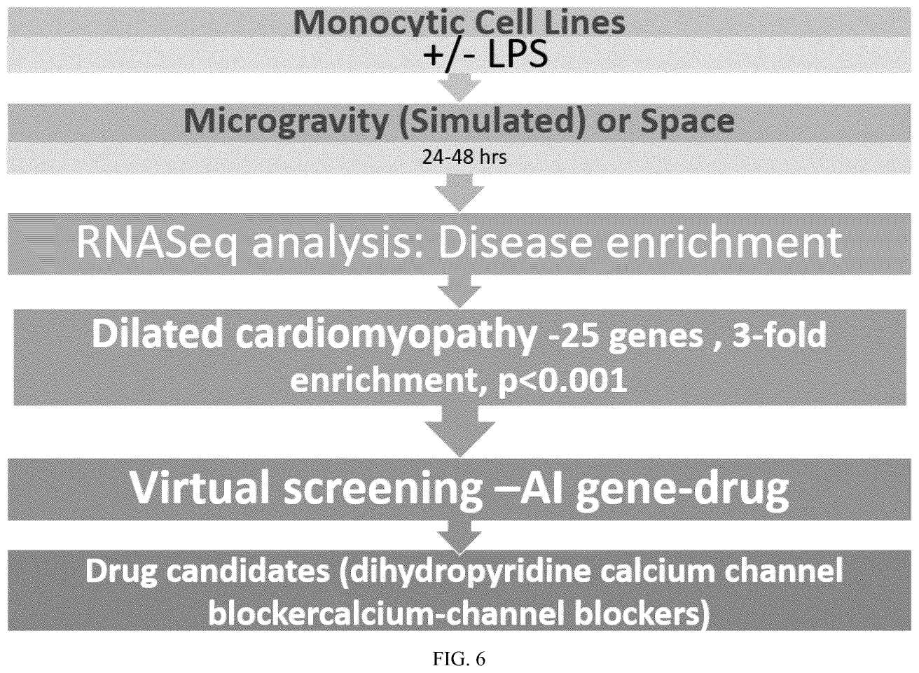

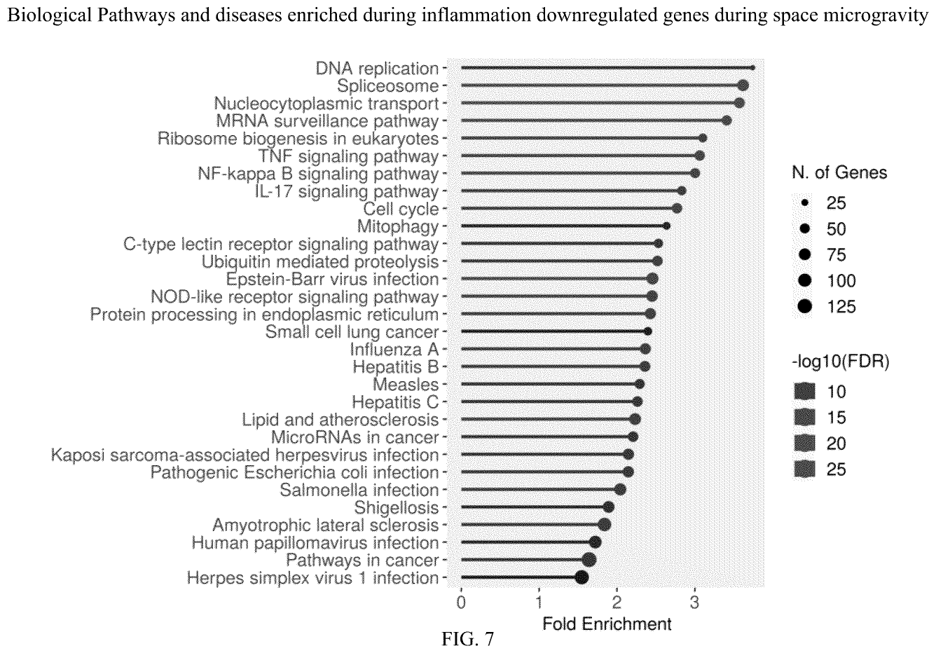

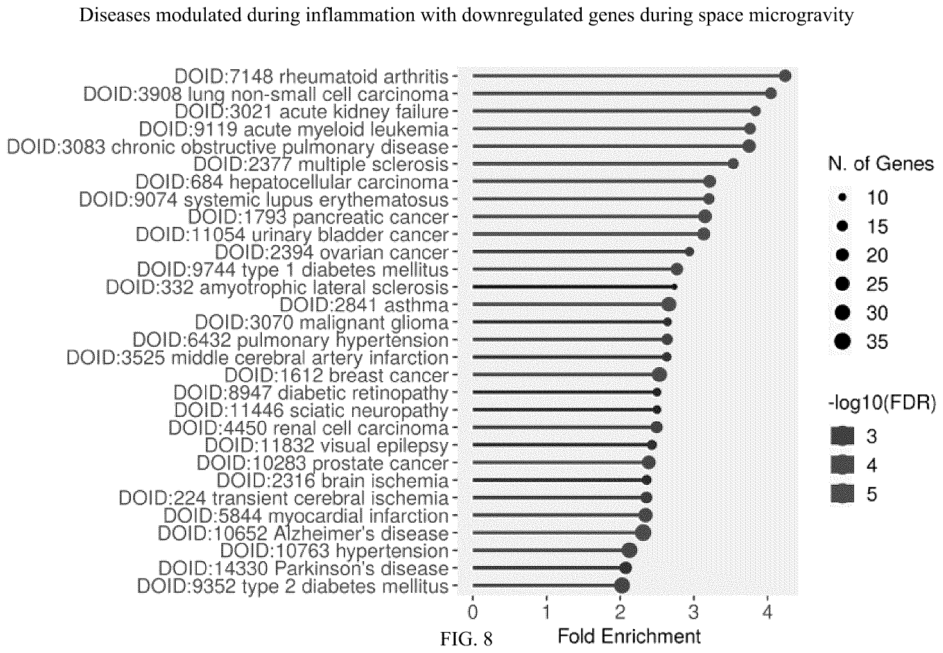

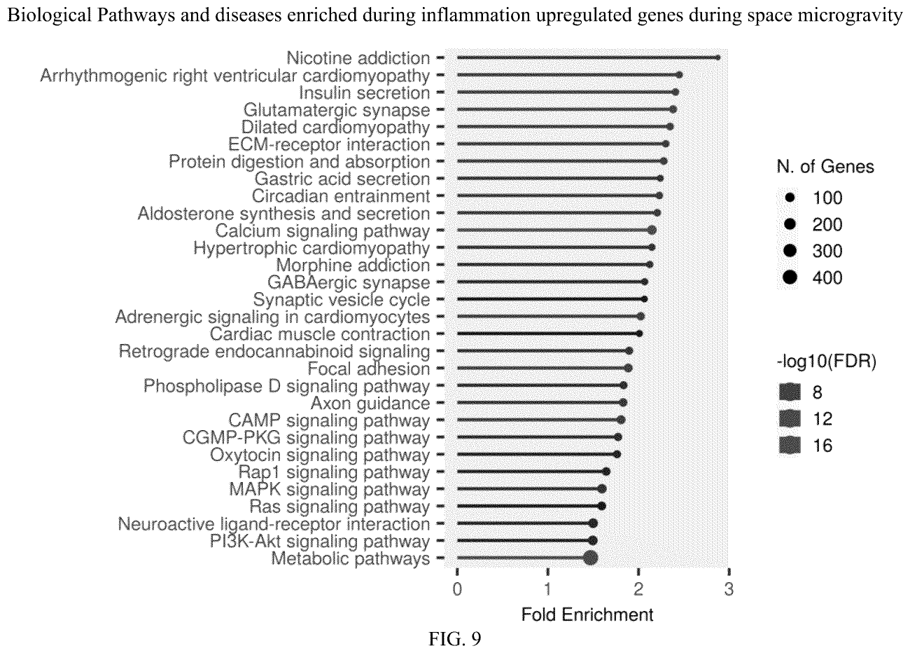

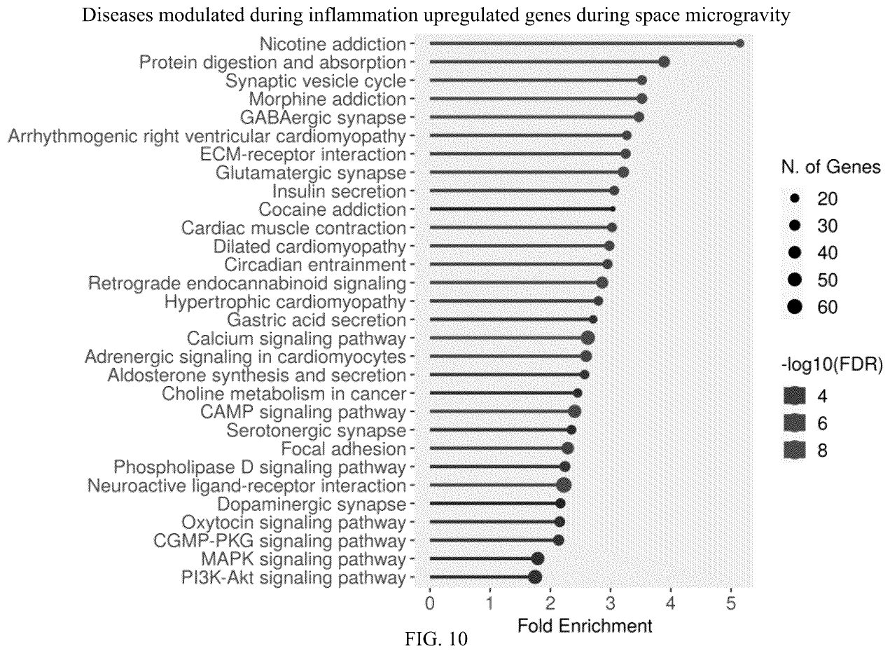

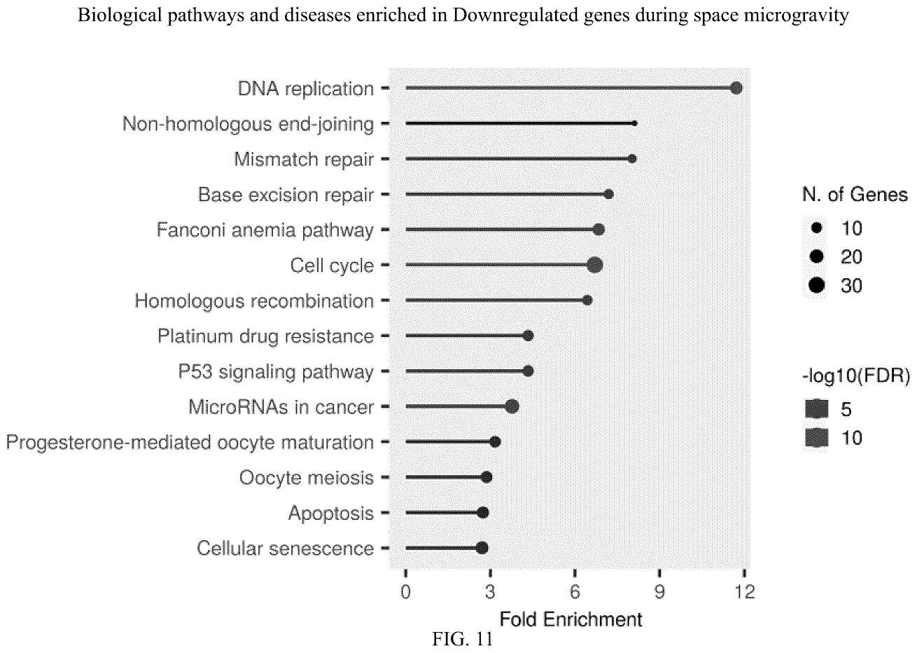

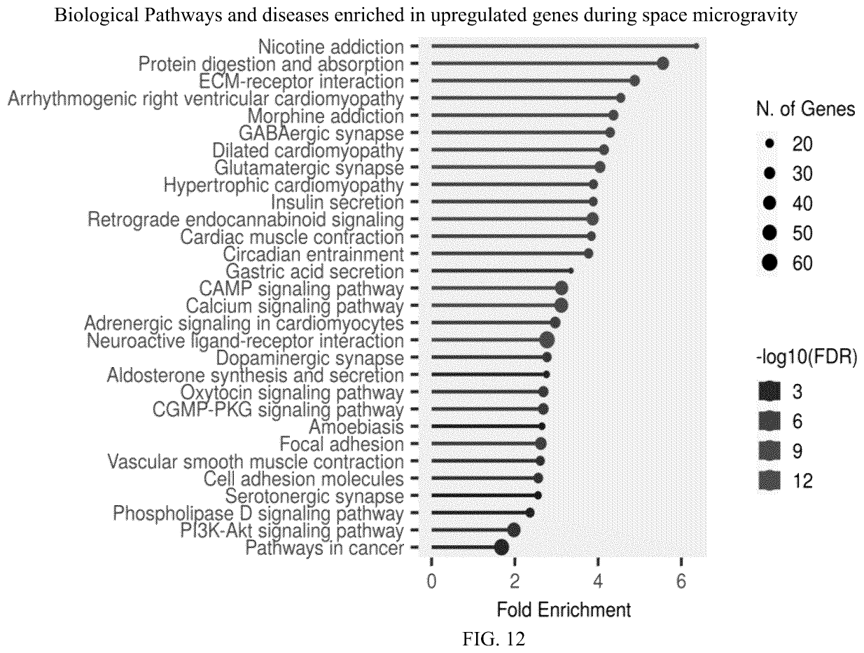

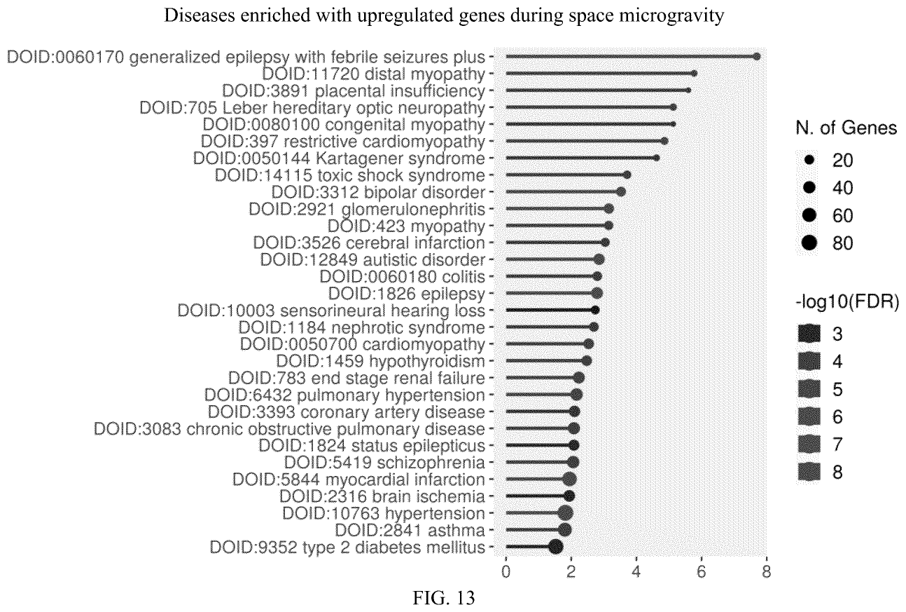

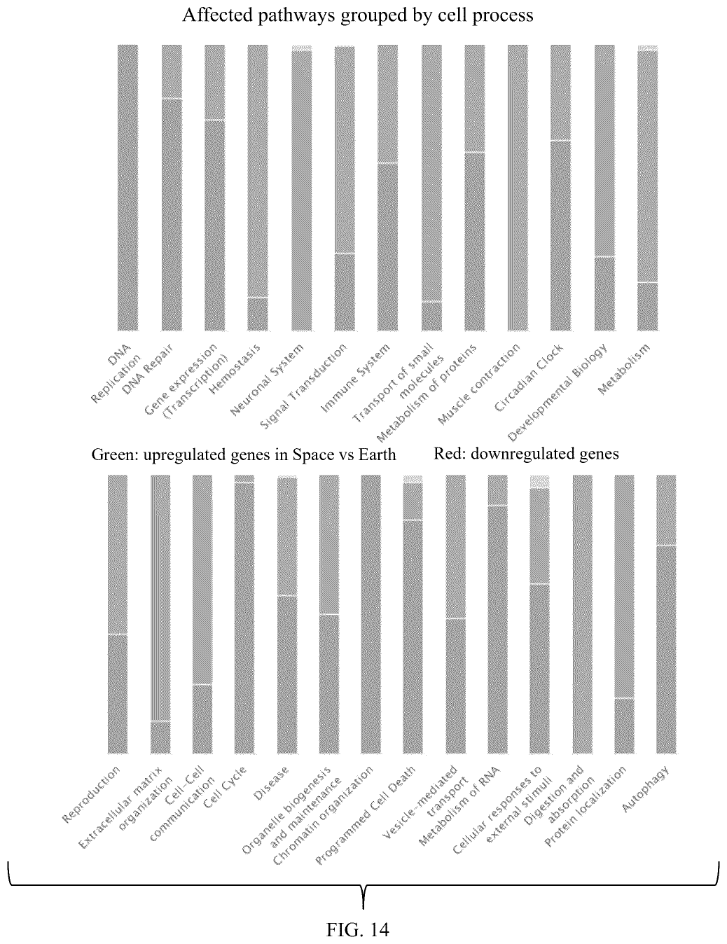

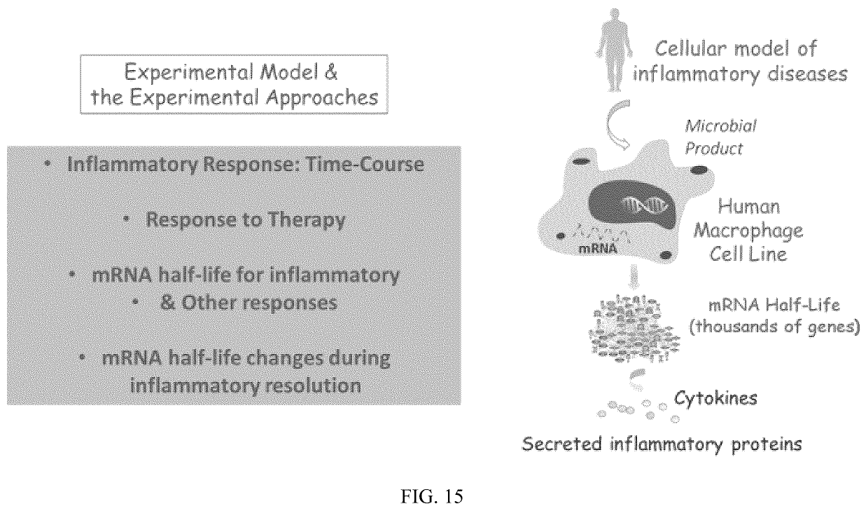

In the following, reference is made to the examples, which are given to illustrate, not to limit the present invention.

### EXAMPLES

The following examples demonstrate the utility of embodiments according to the present invention.

During inflammation, the half-life of a subset of mRNAs-called AU-rich mRNAs-becomes longer leading to further translation and production of pro-inflammatory cytokines. Cytokines and immune mediators participate in several important acute conditions, such as cytokine storm in severe viral diseases, and chronic inflammatory/autoimmune/neurodegenerative diseases.

The exemplary set of experiments outlined herein investigates, inter alia, the inflammatory response (as an example) of human immune cells at microgravity, specifically the changes in mRNA decay, a process that can turn inflammation off. Moreover, response to therapy is mimicked by utilizing the same cellular model. Conditions of microgravity can be generated on earth using specialized equipment, e.g. a specialized plane and flight path, or a rotating wall vessel that has been developed as a microgravity simulator. Alternatively, microgravity conditions may be used as are encountered on board of a space station, such as the ISS. A crew will perform the culturing experiments and take RNA samples for subsequent analysis on ground, where the investigators will monitor RNA expression patterns, and several thousands of mRNA half-lives will be measured. Results contribute to a better understanding of space health and uncover biomarkers or potential therapies for diseases such as inflammatory diseases in both Space and Earth.

Results provide insight into whether microgravity changes the mechanism that turns off inflammation. To the best knowledge of the inventor, researchers have not yet examined mRNA stability and its responsiveness in microgravity. This investigation contributes to a better understanding of disease response, such as inflammatory response in space, helping to maintain astronaut health on future missions, but also providing a disease model system that can be used to identify drugs or drug candidates suitable for treatment. By providing insights into the mechanisms of a disease, e.g. inflammation, this work contributes to development of better prevention and treatment of people experiencing a disease, e.g. inflammation, on Earth.

### Method:

A crew member aboard the International Space Station (ISS) initiates the cell cultures on-orbit in the Life Sciences Glovebox (LSG) by thawing and rinsing the cells using a centrifuge, then seeding the cells into four 48-well plates. The four 48-well plates are incubated at 37° C. and 5% CO2 to give the cultures time to adapt to microgravity. Then, after 48 hours of adaptation time, treatments of varying concentrations are added to the plates inside the LSG. Next, two plates (half of the cultures) are sampled to examine the mRNA half-life at timepoints of 0 h, 0.5 hours, 1 h, 2 h, and 4 h, while the other two plates are a time course experiment with samples taken at 0 h, 1 h, 2 h, 4 h, 6 h, and 22 h. Each half of the plate either receives Dimethyl Sulfoxide (DMSO) carrier or a drug or drug candidate suspected of having a particular pharmacological effect, for example an inhibitor compound suspected of inhibiting a particular disease or pathway. The cell samples are preserved using RNAlater for later RNA extraction and analysis.

The crew collected RNA samples are transferred to Earth for analysis on ground, where the investigators monitor RNA expression patterns, and several thousands of mRNA half-lives are measured.

Initial results were analyzed, using data obtained with culturing cells of the “THP-1 monocytic leukemia cell line” that had been subjected to space microgravity surprisingly led to the following findings:
a) The data (differential gene expression between Earth and Space samples) indicated that samples cultured in space display expression patterns that are indicative of diseases and disease states that are regularly observed in: astronauts.
b) The data indicated that samples cultured in space, hence under conditions of microgravity, display expression patterns that are related to one or several of inflammatory diseases, infectious diseases, cardiovascular diseases, neurological diseases, cancerous diseases, diseases of the kidney, diseases of the lung, and autoimmune diseases, indicating that the model and the methodology of the present invention can be used as a platform-using conditions of microgravity, either simulated microgravity on ground or in space—for studying and discovering disease targets, drug targets, and drugs that potentially alleviate these diseases.

A typical protocol that is representative of embodiments of the present invention can be summarized as follows:
Use a monocytic cell line, such as THP-1, U-937 or KG-1, with or without stimulation for an immune response (using a suitable agent such as an antigen or stimulating agent, preferably selected from lipopolysaccharide (LPS), hemocyanines such as keyhole limpet hemocyanine, phorbol esters such as phorbol myristate acetate (PMA), granulocyte-macrophage colony-stimulating factor (GM-CSF), macrophage colony-stimulating factor (M-CSF), pro-inflammatory cytokines, such as TNFα, IFN-γ, IL-1ß, IL-6, and combinations of the foregoing. Instead of a monocytic cell line, also stimulated peripheral blood mononuclear cells (PBMCs) may be used.
Culture and subject the cells to a condition of a microgravity environment (e.g. as in space or using a simulated microgravity hardware in the lab may also be used instead of true space environment. An example of such hardware is a rotating wall vessel developed by as a microgravity simulator to mimic conditions of microgravity on cells in earth-based laboratory studies (see for example Gardner et al., 2016, Viruses; 8 (11); 304; doi: 10.3390/v8110304)
Incubate the cells with or without a drug or drug candidate-which can be inducing or inhibiting or which is suspected of having a pharmacological effect, for a suitable period, e.g. 6-72 hr under μG (microgravity) conditions vs g (normal gravity) conditions to see the response. Alternatively, one may also compare cells that have been incubated with a drug or drug candidate under microgravity conditions for a suitable period, e.g. 6-72 hr, with cells that have been incubated with a drug or drug candidate under microgravity conditions, but without the drug or drug candidate.
Extract RNA or protein and subject them to differential gene or protein expression using omics approaches such as RNAseq.
Divide the results into functional or disease groups using omics programs. Typically the respective programs already provide suggestions as to what genes are involved in a particular disease or biological pathway.
Determine the cells' response to a drug or drug candidate (or several drugs or drug candidates, by e.g., screening library) for the resultant functional or disease indication.

Results of such experiments are presented in the figures, which show numerous genes that are down- or upregulated under conditions of microgravity, such genes being typical for various diseases, including inflammatory diseases, infectious diseases, cardiovascular diseases, neurological diseases, cancerous diseases, diseases of the kidney, diseases of the lung, and autoimmune diseases; as well as genes being indicative for various biological pathways. Hence, the results show that cell cultures of monocytic cells or stimulated peripheral blood mononuclear cells (PBMCs) which have been cultured under conditions of microgravity, can be used as models for simulating a disease or a biological pathway. Such model can therefore also be used to identify drugs or drug candidates that are suitable for treating or preventing the respective disease or for having an effect on the respective biological pathway.

The results also indicate that there are numerous genes that are down- or upregulated under conditions of microgravity, such genes and/or their up/down-regulation being typical for ageing. Hence, the results show that cell cultures of monocytic cells or stimulated peripheral blood mononuclear cells (PBMCs) which have been cultured under conditions of microgravity, can be used as models for simulating ageing. Such model can therefore also be used to identify drugs or drug candidates that are suitable for influencing ageing, in particular for preventing or delaying the onset or progress of ageing.

The features of the present invention disclosed in the specification, the claims, and/or in the accompanying figures may, both separately and in any combination thereof, be material for realizing the invention in various forms thereof.

1. A method for determining the activity of a drug, or drug candidate, and/or for predicting suitability of a drug, or drug candidate, for treatment or prevention of a disease and/or for predicting suitability of a drug, or drug candidate, for influencing ageing, said method comprising:
a) providing, in any order but separate from each other, a first set of cells of either a monocytic cell line or of stimulated peripheral blood mononuclear cells (PBMCs), and at least one drug or drug candidate;
b) optionally, stimulating an immune response of said first set of cells of said monocytic cell line or of said stimulated peripheral blood mononuclear cells (PBMCs);
c) culturing said first set of cells of said monocytic cell line or of said stimulated PBMCs;
d) subjecting said first set of cells to a condition of microgravity, and exposing said first set of cells of said monocytic cell line or of said stimulated PBMCs to said at least one drug or drug candidate, whilst continuing to subject said first set of cells to a condition of microgravity; or exposing said first set of cells of said monocytic cell line to said at least one drug or drug candidate, and subjecting said first set of cells to a condition of microgravity, whilst continuing to expose said first set of cells of said monocytic cell line to said at least one drug or drug candidate;
e) stopping said first set of cells of said monocytic cell line being subjected to a condition of microgravity;
f) determining RNA expression and/or protein expression of said first set of cells and compare said determined RNA expression and/or said protein expression of said first set of cells with RNA expression and/or protein expression, respectively, of a second set of cells of the same monocytic cell line or stimulated peripheral blood mononuclear cells (PBMCs) that have also been subjected to a condition of microgravity but that have not been exposed to said at least one drug or drug candidate;
wherein differences observed when comparing expression in said first set of cells and said second set of cells, in
expression of RNAs or proteins involved in a biological pathway, and
expression of RNAs or proteins associated with a particular disease, between said first set of cells and said second set of cells, are indicative of said drug or drug candidate being suitable for treatment or prevention of a disease associated with said biological pathway or said particular disease, respectively; and
wherein differences in
expression of RNAs or proteins associated with ageing,
between said first set of cells and said second set of cells, are indicative of said drug or drug candidate being suitable for influencing ageing.

2. The method according to claim 1, wherein said monocytic cell line is a cell line selected from THP-1 monocytic cell line, U-937 monocytic cell line, and KG-1 monocytic cell line; and wherein said stimulated peripheral blood mononuclear cells (PBMCs) are peripheral blood mononuclear cells that have been stimulated by a stimulating agent selected from lipopolysaccharide (LPS), hemocyanines, phorbol esters, granulocyte-macrophage colony-stimulating factor (GM-CSF), macrophage colony-stimulating factor (M-CSF), pro-inflammatory cytokines, and combinations of the foregoing.

3. The method according to claim 1, wherein said method comprises step b).

4. The method according to claim 3, wherein step b) is performed by exposing said first set of cells to an antigen or stimulating agent.

5. The method according to claim 1, wherein said condition of microgravity of step d) is a gravity in the range of from 1×10⁻⁶ g to 1×10⁻² g, with “1 g” being the standard gravity experienced on the surface of Earth.

6. The method according to claim 1, wherein said subjecting said first set of cells to a condition of microgravity in step d) occurs over a period of from 6 hours to 10 days.

7. The method according to claim 1, wherein determining RNA expression of said first set of cells in step f) is performed by RNA-sequencing (RNA-Seq), and wherein determining protein expression of said first set of cells in step f) is performed by mass spectrometry, Olink® proteomics, Somalogic® proteomics, Luminex® proteomics and/or array-type proteomics.

8. The method according to claim 1, wherein said disease is selected from inflammatory diseases, infectious diseases, cardiovascular diseases, neurological diseases, cancerous diseases, diseases of the kidney, diseases of the lung, and autoimmune diseases.

9. The method according to claim 8, wherein
said cardiovascular diseases are selected from coronary artery disease, heart failure, hypertensive heart disease, rheumatic heart disease, cardiomyopathy, dilated cardiomyopathy, myocardial infarction, arrhythmia, congenital heart disease, arteriosclerosis of coronary vessels, valvular heart disease, carditis, aortic aneurysm, peripheral artery disease, thromboembolic disease, venous thrombosis, pulmonary heart disease, endocarditis, and myocarditis;
said neurological diseases are selected from epilepsy, Alzheimer's disease, cerebral infarction, ischemic stroke, multiple sclerosis, Parkinson's disease, amyotrophic lateral sclerosis (ALS), ataxia, and Bell's palsy;
said inflammatory diseases are selected from inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, and inflammatory diseases of the larynx;
said infectious diseases are selected from viral infections, bacterial infections and protozoan infections;
said cancerous diseases are selected from adenocarcinoma, choroidal melanoma, acute leukemia, acoustic neurinoma, ampullary carcinoma, anal carcinoma, astrocytoma, basal cell carcinoma, pancreatic cancer, Desmoid tumor, bladder cancer, bronchial carcinoma, estrogen dependent and independent breast cancer, Burkitt's lymphoma, corpus cancer, Carcinoma unknown primary tumor (CUP-syndrome), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic tumor, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymphomas, stomach cancer, malignant melanoma, malignant neoplasia, malignant tumors gastrointestinal tract, breast carcinoma, rectal cancer, medulloblastomas, melanoma, meningiomas, Hodgkin's/Non-Hodgkin's lymphoma, mycosis fungoides, nasal cancer, neurinoma, neuroblastoma, kidney cancer, renal cell carcinomas, oligodendroglioma, esophageal carcinoma, osteolytic carcinomas and osteoplastic carcinomas, osteosarcomas, ovarian carcinoma, pancreatic carcinoma, penile cancer, plasmacytoma, prostate cancer, pharyngeal cancer, rectal carcinoma, retinoblastoma, vaginal cancer, thyroid carcinoma, esophageal cancer, T-cell lymphoma, thymoma, tube carcinoma, eye tumors, urethral cancer, urologic tumors, urothelial carcinoma, vulva cancer, wart appearance, soft tissue tumors, soft tissue sarcoma, Nephroblastoma, cervical carcinoma, tongue cancer, invasive ductal carcinoma, invasive lobular carcinoma, ductal carcinoma in situ, lobular carcinoma in situ, small-cell lung carcinoma, non-small-cell lung carcinoma, bronchial adenoma, pleuropulmonary blastoma, mesothelioma, brain stem glioma, hypothalamic glioma, cerebellar astrocytoma, cerebral astrocytoma, neuroectodermal tumor, pineal tumors, sarcoma of the uterus, salivary gland cancers, anal gland adenocarcinomas, mast cell tumors, pelvis tumor, ureter tumor, hereditary papillary renal cancers, sporadic papillary renal cancers, intraocular melanoma, hepatocellular carcinoma, cholangiocarcinoma, mixed hepatocellular cholangiocarcinoma, squamous cell carcinoma, malignant melanoma, Merkel cell skin cancer, non-melanoma skin cancer, hypopharyngeal cancer, nasopharyngeal cancer, oropharyngeal cancer, oral cavity cancer, squamous cell cancer, oral melanoma, AIDS-related lymphoma, cutaneous T-cell lymphoma, lymphoma of the central nervous system, malignant fibrous histiocytoma, lymph sarcoma, rhabdomyosarcoma, malignant histiocytosis, fibroblastic sarcoma, hemangiosarcoma, hemangiopericytoma, and leiomyosarcoma (LMS);
said diseases of the kidney are selected from chronic kidney disease, kidney stones, glomerulonephritis, polycystic kidney disease, nephrotic syndrome, end stage renal failure, renal cancer, interstitial nephritis, Fabry disease, focal segmental glomerulosclerosis (FSGS), lupus nephritis, IgA Nephropathy, cystinosis, complement 3 glomerulopathy, atypical hemolytic uremic syndrome, granulomatosis with polyangiitis, cardiovascular-kidney-metabolic syndrome, and minimal change disease;
said diseases of the lung are selected from asthma, chronic obstructive pulmonary disease (COPD), bronchiectasis, bronchitis, pulmonary fibrosis, sarcoidosis, lung cancer, pneumonia, COVID19, pulmonary edema, influenza, and pulmonary hypertension; and
said autoimmune diseases are selected from asthma, diabetes, rheumatic diseases, rejection of transplanted organs and tissues, rhinitis, chronic obstructive pulmonary diseases, osteoporosis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper IgE syndrome (HIES), Wiskott-Aldrich syndrome (WAS), ataxia-telangiectasia, immune mediated cancers, white cell defects, autoimmune diseases, systemic lupus erythematosus (SLE), rheumatoid arthritis (RA), multiple sclerosis (MS), immune-mediated or Type 1 Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, Goodpasture syndrome (GPS), myasthenia gravis (MG), Sympathetic ophthalmia, Phakogene Uveitis, chronical aggressive hepatitis, primary biliary cirrhosis, autoimmune hemolytic anemia, and Werlhof's disease.

10. The method according to claim 1, wherein said influencing is selected from: preventing the onset of ageing, delaying the onset of aging, preventing the progress of ageing, delaying the progress of ageing, and combinations of the foregoing.

11. The method according to claim 3, wherein said antigen or stimulating agent is selected from lipopolysaccharide (LPS), hemocyanines, phorbol esters, granulocyte-macrophage colony-stimulating factor (GM-CSF), macrophage colony-stimulating factor (M-CSF), pro-inflammatory cytokines, and combinations of the foregoing.

12. The method according to claim 9, wherein the inflammatory disease is selected from abscessation, acanthamoeba infection, acne vulgaris, actinomycosis, acute inflammatory dermatoses, acute laryngeal infections of adults, acute multifocal placoid pigment epitheliopathy, acute (thermal) injury, acute retinal necrosis, acute suppurative otitis media, algal disorders, allergic contact dermatitis, amyloidosis angioedema, ankylosing spondylitis, aspergillosis, atopic dermatitis, pseudorabies, autoantibodies in vasculitis, bacterial disorders, bacterial laryngitis, bacterial meningitis, Behçet's disease (BD), birdshot choroidopathy, Gilchrist's disease, Borna disease, brucellosis, bullous myringitis, bursitis, candidiasis, canine distemper encephalomyelitis, canine distemper encephalomyelitis in immature animals, canine hemorrhagic fever, canine herpes virus encephalomyelitis, cholesteatoma, chronic granulomatous diseases (CGD), chronic inflammatory dermatoses, chronic relapsing encephalomyelitis, chronic suppurative otitis media, Ocular Cicatricial pemphigoid (OCP), common upper respiratory infection, granuloma, Crohn's disease, cryptococcal disease, dermatomyositis, diphtheria, discoid lupus erythematosus (DLE), drug-induced vasculitis, drug or hypersensitivity reaction, encephalitozoonosis, eosinophilic meningoencephalitis, Erythema multiforme (EM), feline leukemia virus, feline immunodeficiency virus, feline infectious peritonitis, feline Polioencephalitis, feline spongiform encephalopathy, fibromyalgia, Fuchs Heterochromic Uveitis, gastroesophageal (laryngopharyngeal) reflux disease, giant cell arteritis, glanders, glaucomatocyclitic crisis, gonorrhea granular myringitis, Granulomatous meningoencephalitis (GME), herpes simplex, histoplasmosis, idiopathic diseases, idiopathic inflammatory disorders, immune and idiopathic disorders, infections of the immunocompromised host, infectious canine hepatitis, inhalation laryngitis, interstitial nephritis, irritant contact dermatitis, juvenile rheumatoid arthritis, Kawasaki's disease, La Crosse virus encephalitis, laryngeal abscess, laryngotracheobronchitis, leishmaniasis, lens-induced uveitis, leprosy, leptospirosis, leukemia, lichen planus, lupus, lymphoma, meningitis, meningoencephalitis in greyhounds, miscellaneous meningitis/meningoencephalitis, microscopic polyangiitis, multifocal choroiditis, multifocal distemper encephalomyelitis in mature animals, multiple sclerosis, Muscle Tension Dysphonia (MTD), mycotic (fungal) diseases, mycotic diseases of the CNS, necrotizing encephalitis, neosporosis, old dog encephalitis, onchocerciasis, parasitic encephalomyelitis, parasitic infections, Pars planitis, parvovirus encephalitis, pediatric laryngitis, pollution and inhalant allergy, polymyositis, post-vaccinal canine distemper encephalitis, prion protein induced diseases, protothecosis, protozoal encephalitis-encephalomyelitis, psoriasis, psoriatic arthritis, pug dog encephalitis, radiation injury, radiation laryngitis, radionecrosis, relapsing polychondritis, Reiter's syndrome, retinitis pigmentosa, retinoblastoma, rheumatoid arthritis, Rickettsial disorders, rocky mountain spotted fever, salmon poisoning disease (SPD), Sarcocystosis, sarcoidosis, schistosomiasis, scleroderma, Rhinoscleroma, serpiginous choroiditis, shaker dog disease, Sjogren's syndrome, spasmodic croup, spirochetal (syphilis) diseases, spongiotic dermatitis, sporotrichosis, steroid responsive meningitis-arteritis, Stevens-Johnson syndrome (SJS, EM major), epiglottitis, sympathetic ophthalmia, Syngamosis, syphilis, systemic vasculitis in sarcoidosis, Takayasu's arteritis, tendinitis (tendonitis), Thromboangiitis obliterans (Buerger Disease), tick-borne encephalitis in dogs, toxic epidermal necrolysis (TEN), toxocariasis, toxoplasmosis, trauma, traumatic laryngitis, trichinosis, trypanosomiasis, tuberculosis, tularemia, ulcerative colitis, urticaria (hives), vasculitis, vasculitis and malignancy, vasculitis and rheumatoid arthritis, vasculitis in the idiopathic inflammatory myopathies, vasculitis of the central nervous system, vasculitis secondary to bacterial, fungal, and parasitic infection, viral disorders, viral laryngitis, vitiligo, vocal abuse, vocal-cord hemorrhage, Vogt-Koyanagi-Harada syndrome (VKH), Wegener's granulomatosis, and Whipple's disease.

13. The method according to claim 9, wherein the infectious disease is selected from AIDS, Adenovirus Infection, Alveolar Hydatid Disease (AHD), Amoebiasis, Angiostrongyliasis, Anisakiasis, Anthrax, Babesiosis, Balantidiasis, Baylisascaris Infection, Bilharzia (Schistosomiasis), Blastocystis hominis Infection, Lyme Borreliosis, Botulism, Brainerd Diarrhea, Brucellosis, Bovine Spongiform Encephalopathy (BSE), Candidiasis, Capillariasis, Chronic Fatigue Syndrome (CFS), Chagas Disease, Chickenpox, Chlamydia pneumoniae Infection, Cholera, Chronic Fatigue Syndrome, Creutzfeldt-Jakob Disease (CJD), Clonorchiasis, Cutaneous Larva migrans (CLM), Coccidioidomycosis, Conjunctivitis, Coxsackievirus A16 (Cox A16), Cryptococcal disease, Cryptosporidiosis, West Nile fever, Cyclosporiasis, Neurocysticercosis, Cytomegalovirus Infection, Dengue Fever, Dipylidium caninum Infection, Ebola Hemorrhagic Fever (EHF), Alveolar Echinococcosis (AE), Encephalitis, Entamoeba coli Infection, Entamoeba dispar Infection, Entamoeba hartmanni Infection, Entamoeba polecki Infection, Pinworm Infection, Enterovirus Infection (Polio/Non-Polio), Epstein Barr Virus Infection, Escherichia coli Infection, Foodborne Infection, Aphthae epizooticae, Fungal Dermatitis, Fungal Infections, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis, Lassa Fever, Leishmaniasis, Visceral leishmaniasis (VL), Malaria, Marburg Hemorrhagic Fever, Measles, Meningitis, Mycobacterium avium Complex (MAC) Infection, Naegleria Infection, Nosocomial Infections, Nonpathogenic Intestinal Amebae Infection, Onchocerciasis, Opisthorchiasis, Papilloma virus Infection, Parvovirus Infection, Plague, Pneumocystis Pneumonia (PCP), Polyomavirus Infection, Q Fever, Rabies, Respiratory Syncytial Virus (RSV) Infection, Rheumatic Fever, Rift Valley Fever, Rotavirus Infection, Roundworms Infection, Salmonellosis, Scabies, Shigellosis, Shingles, Sleeping Sickness, Smallpox, Streptococcal Infection, Tapeworm Infection, Tetanus, Toxic Shock Syndrome, Tuberculosis, duodenum, Vibrio parahaemolyticus Infection, Vibrio septicemia, Viral Hemorrhagic Fever, Warts, Waterborne infectious Diseases, Varicella-Zoster Virus infection, Pertussis and Yellow Fever.
